(19) United States
(12) Patent Application Publication
(10) Pub. No.: US 2010/0274195 A1
(43) Pub. Date: Oct. 28, 2010

(54) Implantable Device Suture Bars

(75) Inventors: Patrick J. Eichers, Independence, MN (US); Andrew J. Rennie, Minnetonka, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/711,335

(22) Filed: Feb. 24, 2010

Related U.S. Application Data
(60) Provisional application No. 61/171,888, filed on Apr. 23, 2009.

(51) Int. Cl. A61M 5/00; A61B 17/04

(52) U.S. Cl. 604/175; 606/139

(57) ABSTRACT

A suture bar for facilitating the securing of an implantable medical device in a body. The suture bar including a body member having a length disposed between a first and a second end, the body member being formed of a generally elongate tube that is piercable by a suture needle. The suture bar also including a first and second connector fixedly attached on the first and second end of the body member, the first and second connector operable to connect the implantable medical device to a pocket in a body.

### TECHNICAL FIELD

This disclosure relates to structures for helping to fix an implantable device into an implant pocket. More specifically, the disclosure relates to suture bars for fixing an implantable device into a body.

### BACKGROUND

A variety of medical devices are used for chronic, i.e., long-term, delivery of therapy to patients suffering from a variety of conditions, such as chronic pain, tremor, Parkinson's disease, epilepsy, urinary or fecal incontinence, sexual dysfunction, obesity, spasticity, or gastroparesis. As examples, electrical stimulation generators are used for chronic delivery of electrical stimulation therapies such as cardiac pacing, neurostimulation, muscle stimulation, or the like. Pumps or other fluid delivery devices may be used for chronic delivery of therapeutic agents, such as drugs. Typically, such devices provide therapy continuously or periodically according to parameters contained within a program. A program may comprise respective values for each of a plurality of parameters, specified by a clinician. The devices may be implantable medical devices that receive the program from a programmer controlled by the clinician.

Examples of such implantable medical devices include implantable fluid delivery devices, implantable neurostimulators, implantable cardioverters, implantable cardiac pacemakers, implantable defibrillators, cochlear implants, and others that now exist or may exist in the future. These devices are intended to provide a patient with a therapeutic output to alleviate or assist with a variety of conditions. Typically, such devices are implanted in a patient and provide a therapeutic output under specified conditions on a recurring basis.

One type of implantable medical device (IMD) is an implantable fluid delivery device that delivers a drug, medication or other substance, typically in fluid form, to a patient at a selected therapy site. An implantable fluid delivery device may be implanted at a location in the body of a patient and deliver a fluid through a catheter to a selected delivery site in the body. Drug IMDs, such as implantable fluid delivery pumps, typically include fluid reservoirs that may be self-sealing and may be percutaneously accessible through ports. A fluid delivery device may be configured to deliver a therapeutic agent, such as a drug, from the fluid reservoir to a patient according to a therapy program. The therapy program may specify, for example, the size of a fluid bolus of the therapeutic agent delivered to the patient, the concentration of the therapeutic agent, and/or the delivery rate of the therapeutic agent.

Implantable medical devices are normally sutured into the body of the patient using sutures and suture loops located on the outside of the housing of the medical device. Typically, four suture loops may be positioned on the housing to secure the implantable medical device. However, depending on how the implantable medical device is positioned in the implant pocket, the surgeon implanting the device may not be able to utilize all of the suture loops to secure the medical device. Improvements in structures and methods for securing implantable medical devices are therefore useful.

### SUMMARY

The present description includes a flexible, adaptable suturing aid for securing an implantable medical device into a body.

A suture bar for facilitating the securing of an implantable medical device in a body including a body member having a length disposed between a first and a second end, the body member being formed of a generally elongate tube that is piercable by a suture needle, a first and second connector fixedly attached on the first and second end of the body member, the first and second connector operable to connect the body member to the implantable medical device.

Another aspect is a kit for securing an implantable medical device into a body wherein the kit includes a plurality of suture bars of varying lengths, the suture bars including a body member having a length disposed between a first and a second end, the body member being formed of a generally elongate tube that is piercable by a suture needle, and a plurality of connectors that can be fixedly attached to the first and second end of the body member.

Another aspect is a method of securing an implantable medical device into a body including the steps of providing an implantable medical device, the implantable medical device including at least two suture loops disposed on an outside surface, creating a pocket in a body, securing one or more suture bars to the at least two suture loops of the implantable medical device, and securing the implantable medical device into the pocket in a desired position by suturing the suture bars to the pocket.

### BRIEF DESCRIPTION OF DRAWINGS

FIG. 1 is a conceptual diagram illustrating an example of a fluid delivery system, which includes an implantable medical device that is configured to deliver a therapeutic agent to a patient via a catheter.

FIG. 2 is a conceptual diagram illustrating the placement of an implantable medical device of the present invention.

FIG. 3 is functional block diagram illustrating an example of an implantable fluid delivery device.

FIG. 4 is a functional block diagram illustrating example components of an external programmer for an implantable medical device.

FIG. 5 is a functional block diagram illustrating an implantable medical device with suture bars.

FIG. 6 is a suture bar of the present invention.

### DETAILED DESCRIPTION

Medical devices are useful for treating, managing or otherwise controlling various patient conditions or disorders, such as, but not limited to, pain (e.g., chronic pain, post-operative pain or peripheral and localized pain), tremor, movement disorders (e.g., Parkinson's disease), diabetes, epilepsy, neuralgia, chronic migraines, urinary or fecal incontinence, sexual dysfunction, obesity, gastroparesis, mood disorders, or other disorders. Some medical devices may be configured to deliver one or more therapeutic agents, alone or in combination with other therapies, such as electrical stimulation, to one or more target sites within a patient. For example, in some cases, a medical device may deliver one or more pain-relieving drugs to patients with chronic pain, insulin to a patient with diabetes, or other fluids to patients with different disorders. The medical device may be implanted in the patient for chronic therapy delivery (e.g., longer than a temporary, trial basis) or temporary delivery.

The following detailed description is of the presently contemplated mode of implementing the invention. The embodiment herein is described in terms of an implantable medical device (IMD) that could be any type of device implanted into a body, including, for example, a drug pump, a stimulator, a monitor, a catheter, etc. This description is not to be taken in a limiting sense, but is merely for the purpose of illustrating the general principles of embodiments of the invention. Furthermore, there is no intention to be bound by any theory presented in the preceding background of the invention or the following detailed description of the invention. The scope of the invention is defined by the appended claims.

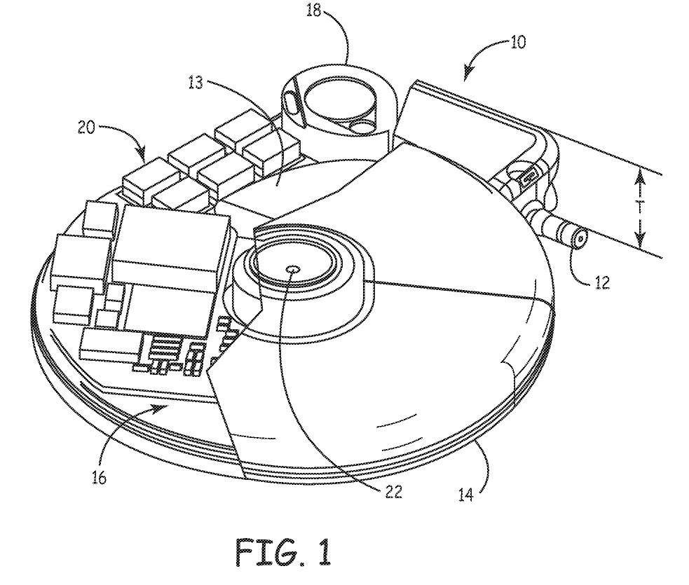

FIG. 1 shows an IMD 10. The illustrated IMD 10 is configured to be surgically implanted into a patient, for example, in the abdominal region, between the skin and the abdominal wall, and is part of a therapy system that may include a catheter connected to the IMD 10. The catheter may deliver infusion medium to the patient, for example, but not limited to, by feeding infusion medium to a particular location in the venous system, within the spinal column, or in the peritoneal cavity of the patient. The therapy system may also include a programmer or other device for controlling the functions of the IMD. For purposes of simplifying the present disclosure, the term “patient” is used herein to refer to any environment in which an implantable device is implanted, whether or not the implant or connection is carried out for medical purposes. The patient may also be referred to by the term “body” to refer to the patient's body. Also, the term “infusion medium” is used herein to refer to any suitable medium delivered by the IMD 10.

The IMD 10 may include a generally disc-shaped housing 14. While a generally circular disc-shaped embodiment is illustrated in FIG. 1, it will be understood that further embodiments of the IMD 10 may employ housing of other shapes, including, but not limited to, oval, oblong, rectangular, or other curved or polygonal shapes. Generally, the housing 14 is made of a biocompatible material and most often has a relatively small diameter and thickness to reduce patient trauma during implant surgery and after implantation. Generally, IMD 10 has an outer housing that is constructed of a biocompatible material that resists corrosion and degradation from bodily fluids, such as titanium or biologically inert polymers.

The housing 14 includes a reservoir 16 for holding a volume of infusion medium, such as, but not limited to, a liquid medication to be administered to the patient. Housing 14 may also contain a drive mechanism 18 (e.g. a pump), a power source 13, and control electronics 20. Pump 18 may be configured to receive infusion media from reservoir 16 via a pump inlet 22. Inlet structure 22 may provide a closeable and sealable fluid flow path to the reservoir in the reservoir portion of the housing. The inlet structure may include a port for receiving a needle through which fluid may be transferred to the IMD, for example, to fill or re-fill the reservoir of the device with the infusion media or a rinsing fluid as will be more fully discussed below. In particular embodiments, the inlet structure may be configured to re-seal after a fill or re-fill operation, and to allow multiple re-fill and re-seal operations. One example of an inlet structure is described in U.S. Pat. No. 6,652,510, titled “Implantable Medical Device and Reservoir for Same,” which is incorporated by reference herein in its entirety and for everything it teaches and discloses. However, further embodiments may employ other suitable inlet structures, including, but not limited to, those described in U.S. Pat. Nos. 5,514,103 and 5,176,644, each to Srisathapat et al.; U.S. Pat. No. 5,167,633 to Mann et al.; U.S. Pat. No. 4,697,622 to Swift; and U.S. Pat. No. 4,573,994 to Fischell et al., also incorporated by reference. Representative examples of reservoir housing portions and reservoirs which may be employed in embodiments of the invention are described in the above referred to U.S. Pat. No. 6,652,510, and further embodiments may employ other suitable reservoir configurations, including, but not limited to, those described in the above referred to U.S. Pat. Nos. 5,514,103; 5,176,644; 5,167,633; 4,697,622; and 4,573,994. The IMD 10 may further include an outlet 12. The outlet 12 illustrated is sized and shaped to connect to a catheter 22 (see below) and to operably attach the catheter 22 to the IMD 10 to receive the therapeutic agent as further discussed below.

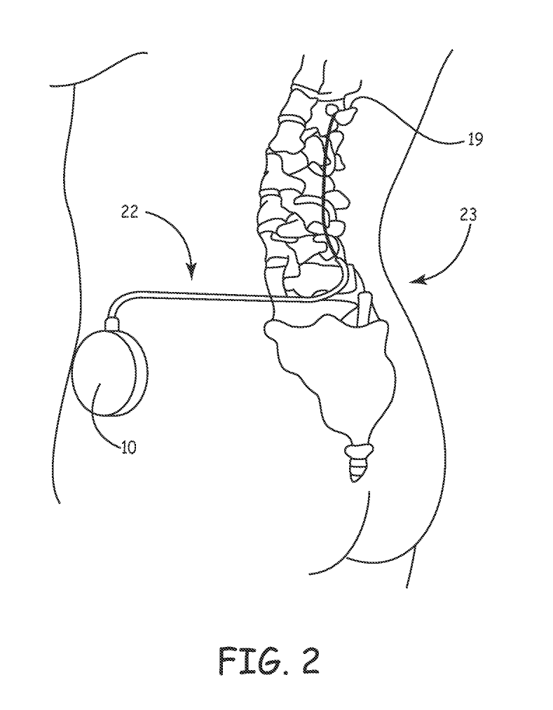

FIG. 2 is a conceptual diagram illustrating an example of a therapy system, which includes IMD 10 configured to deliver at least one therapeutic agent, such as a pharmaceutical agent, insulin, pain relieving agent, anti-inflammatory agent, gene therapy agent, or the like, to a target site within patient 23 via catheter 22, which is coupled to IMD 10. In one example, catheter 22 may comprise a plurality of catheter segments. In the example of FIG. 2, the therapeutic agent is a therapeutic fluid. IMD 10 may be, for example, an implantable fluid delivery device that delivers therapeutic agents in fluid form to patient 23. In the example shown in FIG. 2, the target site is proximate to spinal cord 19 of patient 23. A proximal end of catheter 22 is coupled to IMD 10, while a distal end of catheter 22 is located proximate to the target site. In the present embodiment, therapy system 10 may also include an external programmer 80, which wirelessly communicates with IMD 10 as needed, such as to provide or retrieve therapy information or control aspects of therapy delivery (e.g., modify the therapy parameters, turn IMD 10 on or off, and so forth). Programmer 80 may include a user interface that may display a representation of a portion of an implantable fluid delivery device and simultaneously display an indication of a location of fluid within the implantable fluid delivery device during a delivery phase, as discussed in greater detail below. While patient 23 is generally referred to as a human patient, other mammalian or non-mammalian patients are also contemplated. In other examples, IMD 10 may be implanted within other suitable sites within patient 23, which may depend, for example, on the target site within patient 23 for the delivery of the therapeutic agent.

The IMD 10 and catheter 22 are typically implanted by a clinician (e.g., surgeon) within the body 23 during a surgical procedure. A proximal end of the catheter 22 may be tunneled through the tissue to the IMD 10 location and coupled to a catheter port of the IMD 10. If implanted, the medical device 10 is typically positioned subcutaneously, e.g., from 1 centimeter (0.4 inches) to 2.5 centimeters (1 inch) beneath the skin, where there is sufficient tissue for supporting the IMD 10, e.g., with sutures or the like.

Therapy system may be used, for example, to reduce pain experienced by patient 23. IMD 10 may deliver one or more therapeutic agents to patient 23 according to one or more therapy programs that set forth different therapy parameters, such as bolus size, frequency of bolus delivery, time during which a bolus is to be delivered, and so forth. In some examples, the therapeutic agent may be a liquid. The therapy programs may be may be a part of a program group for therapy, where the group includes a plurality of therapy programs. In some examples, IMD 10 may be configured to deliver a therapeutic agent to patient 23 according to different therapy programs on a selective basis. IMD 10 may include a memory to store one or more therapy programs, instructions defining the extent to which patient 23 may adjust therapy parameters, switch between programs, or undertake other therapy adjustments. Patient 23 may select and/or generate additional therapy programs for use by IMD 10 via external programmer 80 at any time during therapy or as designated by the clinician.

In some examples, multiple catheters 22 may be coupled to IMD 10 to target the same or different tissue or nerve sites within. Thus, although a single catheter 22 is shown in FIG. 1, in other examples, system 12 may include multiple catheters or catheter 22 may define multiple lumens for delivering different therapeutic agents to patient 23 or for delivering a therapeutic agent to different tissue sites within patient 23. Accordingly, in some examples, IMD 10 may include a plurality of reservoirs for storing more than one type of therapeutic agent. In some examples, IMD 10 may include a single long tube that contains the therapeutic agent in place of a reservoir. However, for ease of description, an IMD 10 including a single reservoir is primarily discussed herein with reference to the example of FIG. 1.

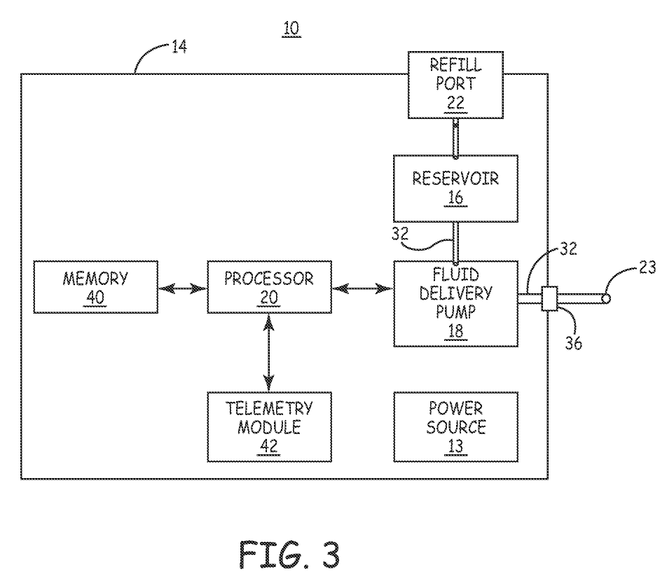

FIG. 3 is a functional block diagram illustrating components of an example of IMD 10, which includes refill port 22, reservoir 16, processor 20, memory 40, telemetry module 42, power source 13, fluid delivery pump 18, internal tubing 32, and catheter access port 36. Fluid delivery pump 18 may be a mechanism that delivers a therapeutic agent in a metered or desired flow rate to the therapy site within patient 23 from reservoir 16 via the catheter 22. Refill port 22 may comprise a self-sealing membrane to prevent loss of therapeutic agent delivered to reservoir 16 via refill port 22. After a delivery system, e.g., a hypodermic needle, penetrates the membrane of refill port 22, the membrane may seal shut when the needle is removed from refill port 22.

Internal tubing 32 is a segment of tubing that runs from reservoir 16, around or through fluid delivery pump 18, to catheter access port 36. In one example, fluid delivery pump 18 may be a squeeze pump that squeezes internal tubing 32 in a controlled manner, e.g., such as a peristaltic pump, to progressively move fluid from reservoir 16 to the distal end of catheter 22 and then into the patient 23 according to parameters specified by a set of program information. Fluid delivery pump 18 may, in other examples, comprise an axial pump, a centrifugal pump, a pusher plate, a piston-driven pump, or other means for moving fluid through internal tubing 32 and catheter 22.

Processor 20 controls the operation of fluid delivery pump 18 with the aid of program information stored in memory 40. For example, the program information may include instructions that define therapy programs to specify the amount of a therapeutic agent that is delivered to a target tissue site within patient 23 from reservoir 16 via catheter 22. The instructions may further specify the time at which a bolus will be delivered and the time interval over which the bolus will be delivered, e.g., as defined by a start and an end time. The therapy programs may also include other therapy parameters, such as the frequency of bolus delivery, the type of therapeutic agent delivered (if IMD 10 is configured to deliver more than one type of therapeutic agent), and so forth. Components described as processors within IMD 10, external programmer 80, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination.

Memory 40 may include any volatile or non-volatile media, such as a random access memory (RAM), read only memory (ROM), non-volatile RAM (NVRAM), electrically erasable programmable ROM (EEPROM), flash memory, and the like. As mentioned above, memory 40 may store program information including instructions for execution by processor 20, such as, but not limited to, therapy programs, historical therapy programs, timing programs for delivery of fluid from reservoir 16 to catheter 22, and any other information regarding therapy of patient 23. A program may indicate the bolus size or flow rate of the drug, and processor 20 may accordingly deliver therapy. Memory 40 may include separate memories for storing instructions, patient information, therapy parameters (e.g., grouped into sets referred to as “therapy programs”), therapy adjustment information, program histories, and other categories of information such as any other data that may benefit from separate physical memory modules. Therapy adjustment information may include information relating to timing, frequency, rates and amounts of patient boluses or other permitted patient modifications to therapy. In some examples, memory 40 stores program instructions that, when executed by processor 20, cause IMD 10 and processor 20 to perform the functions attributed to them in this disclosure.

Telemetry module 42 in IMD 10, as well as telemetry modules in other devices described herein, such as programmer 80, may accomplish communication by RF communication techniques. In other embodiments, telemetry module 42 may communicate with the programmer 80 in other methods, such as, for instance, telemetry module 42 may communicate with programmer 80 via proximal inductive interaction. Accordingly, telemetry module 42 may send information to external programmer 80 on a continuous basis, at periodic intervals, or upon request from the programmer. Processor 20 controls telemetry module 42 to send and receive information.

Power source 13 delivers operating power to various components of IMD 10 (connection lines not shown). Power source 13 may include a small rechargeable or non-rechargeable battery and a power generation circuit to produce the operating power. In the case of a rechargeable battery, recharging may be accomplished through proximal inductive interaction between an external charger and an inductive charging coil within IMD 10. In other embodiments, power requirements may be small enough to allow IMD 10 to utilize patient motion and implement a kinetic energy-scavenging device to trickle charge a rechargeable battery. In other examples, traditional batteries may be used for a limited period of time. As a further alternative, an external inductive power supply could transcutaneously power IMD 10 whenever measurements are needed or desired.

Programmer 80, as further discussed and detailed herein, may be an external computing device that is configured to wirelessly communicate with IMD 10. For example, programmer 80 may be a clinician programmer that the clinician uses to communicate with IMD 10. Alternatively, programmer 80 may be a patient programmer that allows patient 23 to view and modify therapy parameters. The clinician programmer may include additional or alternative programming features than the patient programmer. For example, more complex or sensitive tasks may only be allowed by the clinician programmer to prevent patient 23 from making undesired changes to the operation of IMD 10.

Programmer 80 may be a hand-held computing device that includes a display viewable by the user and a user input mechanism that can be used to provide input to programmer 80. For example, programmer 80 may include a small display screen (e.g., a liquid crystal display or a light emitting diode display) that presents information to the user. In addition, programmer 80 may include a keypad, buttons, a peripheral pointing device, touch screen or another input mechanism that allows the user to navigate though the user interface of programmer 80 and provide input.

If programmer 80 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 80 may be a touch screen that allows the user to provide input directly to the user interface shown on the display. The user may use a stylus or their finger to provide input to the display.

In other examples, rather than being a handheld computing device or a dedicated computing device, programmer 80 may be a larger workstation or a separate application within another multi-function device. For example, the multi-function device may be a cellular phone, personal computer, laptop, workstation computer, or personal digital assistant that can be configured to an application to simulate programmer 80. Alternatively, a notebook computer, tablet computer, or other personal computer may enter an application to become programmer 80 with a wireless adapter connected to the personal computer for communicating with IMD 10.

When programmer 80 is configured for use by the clinician, programmer 80 may be used to transmit initial programming information to IMD 10. This initial information may include hardware information for system 10 such as the type of catheter 22, the position of catheter 22 within patient 23, the type of therapeutic agent(s) delivered by IMD 10, a baseline orientation of at least a portion of IMD 10 relative to a reference point, therapy parameters of therapy programs stored within IMD 10 or within programmer 80, and any other information the clinician desires to program into IMD 10.

Whether programmer 80 is configured for clinician or patient use, programmer 80 may communicate to IMD 10 or any other computing device via wireless communication. Programmer 80, for example, may communicate via wireless communication with IMD 10 using radio frequency (RF) telemetry techniques known in the art. Programmer 80 may also communicate with another programmer or computing device via a wired or wireless connection using any of a variety of local wireless communication techniques, such as RF communication according to the 802.11 or Bluetooth specification sets, infrared (IR) communication according to the IRDA specification set, or other standard or proprietary telemetry protocols. Programmer 80 may also communicate with another programming or computing device via exchange of removable media, such as magnetic or optical disks, or memory cards or sticks. Further, programmer 80 may communicate with IMD 10 and another programmer via remote telemetry techniques known in the art, communicating via a local area network (LAN), wide area network (WAN), public switched telephone network (PSTN), or cellular telephone network, for example.

In other applications of therapy system 10, the target therapy delivery site within patient 23 may be a location proximate to sacral nerves (e.g., the S2, S3, or S4 sacral nerves) in patient 23 or any other suitable nerve, organ, muscle or muscle group in patient 23, which may be selected based on, for example, a patient condition. For example, therapy system 10 may be used to deliver a therapeutic agent to tissue proximate to a pudendal nerve, a perineal nerve or other areas of the nervous system. In some instances catheter 22 may be implanted and substantially fixed proximate to the respective nerve. As further examples, catheter 22 may be positioned to deliver a therapeutic agent to help manage peripheral neuropathy or post-operative pain mitigation, ilioinguinal nerve therapy, intercostal nerve therapy, gastric stimulation for the treatment of gastric motility disorders and/or obesity, muscle stimulation, for mitigation of other peripheral and localized pain (e.g., leg pain or back pain). As another example, catheter 22 may be positioned to deliver a therapeutic agent to a deep brain site or within the heart (e.g., intraventricular delivery of the agent). Delivery of a therapeutic agent within the brain may help manage any number of disorders or diseases. Example disorders may include depression or other mood disorders, dementia, obsessive-compulsive disorder, migraines, obesity, and movement disorders, such as Parkinson's disease, spasticity, and epilepsy. Catheter 22 may also be positioned to deliver insulin to a patient with diabetes.

Examples of therapeutic agents that IMD 10 may be configured to deliver include, but are not limited to, insulin, morphine, hydromorphone, bupivacaine, clonidine, other analgesics, genetic agents, antibiotics, nutritional fluids, analgesics, hormones or hormonal drugs, gene therapy drugs, anticoagulants, cardiovascular medications or chemotherapeutics. Various embodiments of the present invention may be utilized with any type of medical device that is to be implanted into the body to aid in securing the medical device into the desired position.

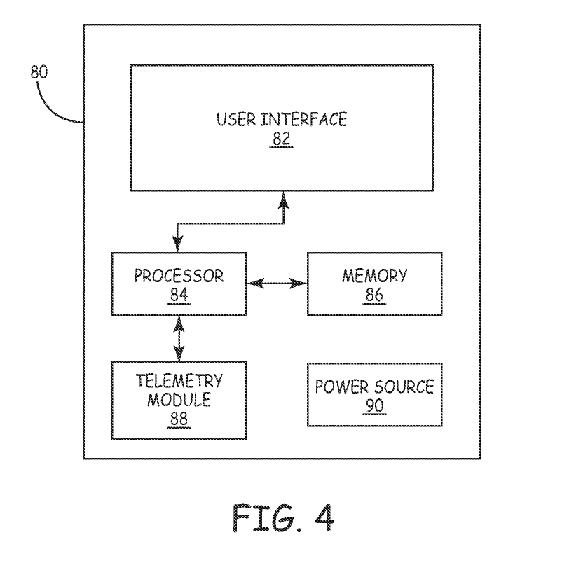

FIG. 4 is a functional block diagram illustrating various components of one example external programmer 80 for IMD 10. As shown in FIG. 4, external programmer 80 includes processor 84, memory 86, telemetry module 88, user interface 82, and power source 90. A clinician or patient 23 interacts with user interface 82 in order to manually change the parameters of a program, change programs within a group of programs, view therapy information, view historical therapy regimens, establish new therapy regimens, or otherwise communicate with IMD 10 or view programming information.

User interface 82 may include a screen and one or more input buttons, as discussed in greater detail below, that allow external programmer 80 to receive input from a user. Alternatively, user interface 82 may additionally or only utilize a touch screen display, as in the example of clinician programmer 60. The screen may be a liquid crystal display (LCD), dot matrix display, organic light-emitting diode (OLED) display, touch screen, or any other device capable of delivering and/or accepting information. For visible indications of therapy program parameters or operational status, a display screen may suffice. For audible and/or tactile indications of therapy program parameters or operational status, programmer 80 may further include one or more audio speakers, voice synthesizer chips, piezoelectric buzzers, or the like.

Input buttons for user interface 82 may include a touch pad, increase and decrease buttons, emergency shut off button, and other buttons needed to control the therapy, as described above with regard to patient programmer 80. Processor 84 controls user interface 82, retrieves data from memory 86 and stores data within memory 86. Processor 84 also controls the transmission of data through telemetry module 88 to IMD 10. The transmitted data may include therapy program information specifying various drug delivery program parameters. Memory 86 may include operational instructions for processor 84 and data related to therapy for patient 23. User interface 82 may be configured to present therapy program information to the user. User interface 82 enables a user to program IMD 10 in accordance with one or more therapy delivery programs, schedules, or the like. The therapy program information may also be stored within memory 86 periodically during therapy, whenever external programmer 80 communicates within IMD 10, or only when the user desires to use or update the therapy program information.

Telemetry module 88 allows the transfer of data to and from IMD 10. Telemetry module 88 may communicate automatically with IMD 10 at a scheduled time or when the telemetry module detects the proximity of IMD 10. Alternatively, telemetry module 88 may communicate with IMD 10 when signaled by a user through user interface 82. To support RF communication, telemetry module 88 may include appropriate electronic components, such as amplifiers, filters, mixers, encoders, decoders, and the like. Power source 90 may be a rechargeable battery, such as a lithium ion or nickel metal hydride battery. Other rechargeable or conventional batteries may also be used. In some cases, external programmer 80 may be used when coupled to an alternating current (AC) outlet, i.e., AC line power, either directly or via an AC/DC adapter.

In some examples, external programmer 80 may be configured to recharge IMD 10 in addition to programming IMD 10. Alternatively, a recharging device may be capable of communication with IMD 10. Then, the recharging device may be able to transfer programming information, data, or any other information described herein to IMD 10. In this manner, the recharging device may be able to act as an intermediary communication device between external programmer 80 and IMD 10. The techniques described in this disclosure may be communicated between IMD 10 via any type of external device capable of communication with IMD 10.

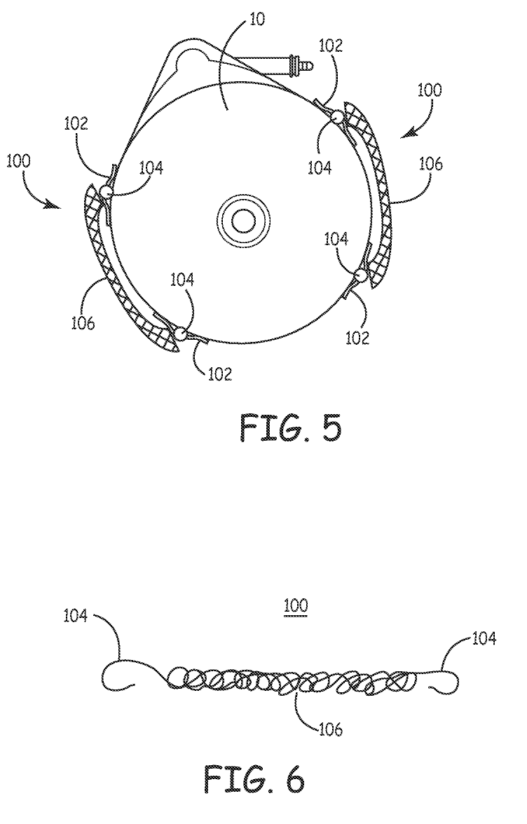

FIG. 5 illustrates an IMD 10 with suture bars 100 for securing the IMD 10 into a patient 23. The suture bar 100 may include a generally elongate body 106 and at least one suture bar connector 104 attached at the ends of the body 106. In the illustrated embodiment, the suture bar 100 is secured to the IMD 10 by attaching the suture bar connectors 104 to existing suture loops 102. As illustrated, the suture bar connector 104 is shown as a generic attachment device and is further described in detail below.

The body 106 of the suture bar 100 may be made of any biocompatible polymer material that is known to those of skill in the art such as, for example, polyethylene terephthalate (PET). The biocompatible polymer may be molded or extruded. Other suitable material may be material that is used in making suture materials, such as polypropylene, polyester, or nylon. Other materials may have various properties as desired, such as being elastic. Elastic materials may include copolymers of styrene-butadiene, polybutadiene, polymers formed from ethylene-propylene diene monomers, polychloroprene, polyisoprene, copolymers of acrylonitrile and butadiene, copolymers of isobutyldiene and isoprene, polyurethanes and the like. In further embodiments, as discussed below, absorbable suture material may also be used.

In the embodiment shown in FIG. 6, the body 106 of the suture bar 100 is constructed as a mesh formed into a hollow tube. The body 106 may have a cross sectional area that allows for sutures to be placed there through. The sutures placed through the body 106 of the suture bar 100 allow for the IMD 10 to be secured into the desired location using a series of spaced apart sutures, or sutures in a line, rather than sutures at specific points as required by utilization of the suture loops 102.

As illustrated, the body 106 of the suture bar 100 is generally a hollow elongate cylindrical shape. In various embodiments, the suture bar 100 may be of a uniform thickness or may have varying thicknesses along its length. For example, the middle of the body 106 may have a slightly thicker width so as to facilitate easy piercing during implantation. The ends of the body 106 may be slightly narrower to reduce overall volume of the suture bar 100. In still further embodiments the body 106 of the suture bar 106 can have several variations in width along its length in order to reduce size, weight, or to provide easier suturing. In still further embodiments the suture bar may be solid or comprised of a tightly woven three dimensional mesh. As may be appreciated, the body 106 of the suture bar may have a generally open structure (a loosely woven mesh) or a generally closed structure (tightly woven mesh) as is desired. In still further embodiments the body 106 may be braided.

The suture bar 100 may be of a length designed to fit along the IMD 10 between successive suture loops 102 or may connect to three or more suture loops. The length of the body 106 of the suture bar 100 and the distance between the suture loops 102 may be balanced to provide a desired tension in the body 106 of the suture bar 100 after it is connected to the suture loops 102. If the suture bar 100 is too short, attaching the suture bar to the suture loops 102 may be difficult.

As may be appreciated, the suture bar 100 provides improved implantation stability for the IMD 10. The suture bar 100 may provide an increased suture area for the clinician (surgeon) to attach the IMD 10 to the implant pocket. The suture bar 100 may free the clinician from only having four distinct points in which to place sutures to secure the IMD 10. In further embodiments, the suture bar 100 may also be designed to promote tissue in-growth after implantation of the IMD 10. Tissue in-growth may further secure the IMD 10 in the implant pocket and reduce surgical revisions that may be necessary to correct a flipped or migrated IMD 10.

The suture bar connector 104 may be a molded clip such as is illustrated in FIG. 6. Such a clip may be made out of a plastic, metal, silicon or any other material that is compatible with medical devices and the implantation of medical devices. It may be desirable to minimize sharp points, edges, or surfaces that can cause irritation. The suture bar connector 104 may be attached to the suture bar 100 by weaving, tying, welding, sonic welding, or by any other attachment method compatible with the material of the suture bar connector 104 and the suture bar 100.

In further embodiments the suture bar connector 104 may be any type of clip or connector known to those of skill in the art that can be secured to or through the suture loop 102, such as, for example a snap clip, a spring clip, a single-sided arrowhead, a flexible wedge, a flexible tie or twist tie, etc. In other embodiments, the suture loop 102 may be replaced by another structure that corresponds to the suture bar connector 104 whereby the suture loop 102 and the suture bar connector 102 are any corresponding connectors for creating a link. In still further embodiments the suture bar 100 may be directly sewn or sutured to the suture loop 102 before the IMD 10 is placed into the desired position, wherein afterwards the suture bar 100 is utilized to fix the IMD 10 in place. In such an embodiment each end of the suture body 106 may be reinforced so as to provide the necessary strength to secure the suture bar 100 and the IMD 10 after implantation.

In still further embodiments the body 106 of the suture bar 100 may be made of a substantially inelastic or elastic cord that can be penetrated by a suture needle. In still further embodiments that body 106 may be made of an extruded plastic material such that the body 106 is a relatively solid piece that is of a desired durometer and that can both be pierced by a suture needle and retain the suture thread. In still further embodiments the suture bar 100 may be constructed of material that is completely or substantially resorbable. Such suture bars 100 may be constructed such that long term tissue in-growth keeps the IMD 10 secured after the suture bar 100 is eroded. In further embodiments the suture bar 100 may be made of materials that are not resorbable.

In further embodiments the suture bar 100 may incorporate radiopaque materials in order to be visible through standard imaging methods.

In further embodiments, the suture bar 100 may also provide a location for dispensing a therapeutic agent. Such materials may include antibiotic, antiviral, antiseptic, anti-infective, or other therapeutic agents or pharmaceuticals that can be eluted from a polymer or other material incorporated into the suture bar 100. Such materials may help to reduce infections or other physiological reactions after the IMD 10 is implanted. In further embodiments, the suture bar 100 may incorporate a pouch or other pocket that allows for a desired material to be loaded into the suture bar 100 before placement by the clinician. As may be appreciated, the location of the pocket should not interfere with the primary purpose of providing an area to secure the IMD 10 during implantation. Further, the therapeutic agent should be selected to be compatible with the material forming the suture bar 100 and the suture bar connector 104.

In still further embodiments the length of the suture bar 100 may be adjusted by the clinician during the implantation procedure. The length may be adjusted by stretching, uncoiling, or cutting the suture bar 100. In further embodiments the body 106 may be a woven or braided mesh that includes a pre-tied sliding knot that can be secured in a manner to result in a desired final length of the body 106. In such embodiments the suture bar connector 104 may be attached to the suture bar 100 after the suture bar is trimmed to the desired length. In still further embodiments, the connector 104 may be utilized to adjust the overall length of suture bar 100 to provide the desired tension between the suture loops 102.

While at least one exemplary embodiment has been presented in the foregoing detailed description of the invention, it should be appreciated that a vast number of variations exist. It should also be appreciated that the exemplary embodiment or exemplary embodiments are only examples and are not intended to limit the scope, applicability, or configuration of the invention in any way. Rather, the foregoing detailed description will provide those skilled in the art with a convenient road map for implementing exemplary embodiments of the invention, it being understood that various changes may be made in the function and arrangement of elements described in an exemplary embodiment without departing from the scope of the invention as set forth in the appended claims and their legal equivalents.

1. A suture bar for facilitating the securing of an implantable medical device in a body, comprising:
a body member having a length disposed between a first and a second end, the body member being formed of a generally elongate tube that is piercable by a suture needle;
a first and second connector fixedly attached on the first and second end of the body member, the first and second connector operable to connect the body member to the implantable medical device.

2. The suture bar of claim 1 wherein the body member is constructed of a mesh material.

3. The suture bar of claim 1 wherein the body member is made of a three dimensional mesh.

4. The suture bar of claim 3 wherein the mesh is a tightly woven mesh.

5. The suture bar of claim 1 wherein the suture bar is made of one or more of polyethylene terephthalate (PET), polypropylene, polyester, nylon, copolymers of styrene-butadiene, polybutadiene, polymers formed from ethylene-propylene diene monomers, polychloroprene, polyisoprene, copolymers of acrylonitrile and butadiene, copolymers of isobutyldiene and isoprene, and polyurethane.

6. The suture bar of claim 1 wherein the first and second connectors are one or more of a snap clip, a spring clip, a single-sided arrowhead, or a flexible wedge.

7. The suture bar of claim 1 wherein the body member is bioabsorbable.

8. The suture bar of claim 1 further comprising a therapeutic agent releasable from the body member when implanted in a patient.

9. The suture bar of claim 1 wherein the length of the body member is adjustable.

10. A kit for securing an implantable medical device into a body, comprising:
a plurality of suture bars of varying lengths, the suture bars including a body member having a length disposed between a first and a second end, the body member being formed of a generally elongate tube that is piercable by a suture needle; and
a plurality of connectors that can be fixedly attached to the first and second end of the body member.

11. The kit of claim 10 wherein the body member is constructed of a mesh material.

12. The kit of claim 10 wherein the body member is made of a three dimensional mesh.

13. The kit of claim 10 wherein the mesh is a tightly woven mesh.

14. The kit of claim 10 wherein the suture bar is made of one or more of polyethylene terephthalate (PET), polypropylene, polyester, nylon, copolymers of styrene-butadiene, polybutadiene, polymers formed from ethylene-propylene diene monomers, polychloroprene, polyisoprene, copolymers of acrylonitrile and butadiene, copolymers of isobutyldiene and isoprene, and polyurethane.

15. The kit of claim 10 wherein the first and second connectors are one or more of a snap clip, a spring clip, a single-sided arrowhead, or a flexible wedge.

16. The kit of claim 10 wherein the body member is bioabsorbable.

17. The kit of claim 10 further comprising a therapeutic agent releasable from the body member when implanted in a patient.

18. A method of securing an implantable medical device into a body comprising:
providing an implantable medical device, the implantable medical device including at least two suture loops disposed on an outside surface;
creating a pocket in a body;
securing one or more suture bars to the at least two suture loops of the implantable medical device; and
securing the implantable medical device into the pocket in a desired position by suturing the suture bars to the pocket.
